(19) United States
(12) Plant Patent Application Publication
(10) Pub. No.: US 2007/0143890 P1
(43) Pub. Date: Jun. 21, 2007

(54) Blueberry named 'Robeson'

(75) Inventors: James Ralph Ballington, Cary, NC (US); Susan Duke Rooks, Rock Point, NC (US)

Correspondence Address: James R. Ballington, Campus Box 7609 / Kilgore Hall, Raleigh, NC, 27695-7609, US

(73) Assignee: North Carolina State University, Raleigh, NC (US)

(21) Appl. No.: 11/303,720

(22) Filed: Dec. 19, 2005

(51) Int. Cl. A01H 5/00

(52) U.S. Cl. PLT/203

(57) ABSTRACT

Vaccinium×‘Robeson’ is a new and distinct pentaploid variety of blueberry that has the following unique combination of desirable features that are outstanding in a new variety. 1. Early ripening compared to hexaploid varieties. 2. Consistent high yields of medium size fruit best suited for local direct marketing. 3. Very good fruit quality and good color 4. Very vigorous plants with upright plant habit and broad soil adaptation, including higher pH soils.

### LATIN NAME OF THE GENUS AND SPECIES

The Latin name of the novel blueberry variety disclosed herein is Vaccinium×‘Robeson’.

### VARIETY DENOMINATION

The inventive hybrid Vaccinium cultivar disclosed herein is pentaploid with 2n=5X=60 chromosomes and has been given the variety denomination ‘Robeson’.

### BACKGROUND OF THE INVENTION

The present invention relates to a new and distinct pentaploid hybrid Vaccinium (blueberry) cultivar grown as a fruiting woody shrub for commercial agriculture. Blueberries are typically consumed both fresh and in a number of processed products.

The new and distinct pentaploid hybrid variety of blueberry (Vaccinium Linnaeus) originated from the hand pollinated cross of ‘US 226’ [tetraploid (2n=4x=48 chromosomes), a hybrid of Vaccinium corymbosum Linnaeus×V. myrtilloides Michaux] (unpatented)×‘Premier’ {hexaploid (2n=6X=72 chromosomes) [(V. virgatum Aiton (syn. V. ashei Reade)]} (unpatented) made in 1980 at the North Carolina State University greenhouses in Raleigh, N.C.

Seeds from this hand pollination were germinated in winter 1980/1981 in Raleigh, N.C., and 35 seedlings were established on the North Carolina State University Research Station at Jackson Springs, N.C., in 1982. When the seedlings reached maturity in 1986, an elite genotype designated as ‘NC 2849’ was selected for its vigorous erect plant habit, productivity, fruit size, color, picking scar, quality and early maturity by James R. Ballington. N.C. 2849 was by far the superior genotype of the two seedlings in the progeny of 35 that produced attractive blue fruit.

During 1987 the original seedling of NC 2849 was propagated by softwood stem cuttings, and following rooting, single three plant plots were established on North Carolina State University Agricultural Research Service experiment stations located at Jackson Springs and Fletcher, N.C., in 1988. Based on its performance in these initial trials, in 1996 it was propagated again by stem cuttings and established in a replicated trial at the Castle Hayne Research Station, Castle Hayne, N.C. It was also established in a grower trial at Ivanhoe N.C., under a Memorandum of Agreement whereby the grower provided the land and care of the plants and North Carolina State University retained ownership of the variety. Plants and fruit of this new variety have remained true to type through these successive cycles of asexual propagation. This new variety has been named the ‘Robeson’ cultivar.

### SUMMARY OF THE INVENTION

‘Robeson’ is a new and distinct pentaploid blueberry variety that begins ripening somewhat earlier than its ‘Premier’ parent. The plant is very vigorous and upright with very broad soil adaptation, including higher pH soils. Yield is good and similar to Premier. Fruit size is medium but firmness only average so that it is not adaptable to mechanical harvesting for the fresh market. Fruit quality is very good and color good. Short shelf-life of fruit indicates that it is best suited for local direct marketing outlets. Leaves are narrowly elliptic-obovate in shape and both the leaf base and apex angles acute. The flowers are not self-fertile and produce very little viable pollen. The corolla of individual flowers is white and cylindro-urceolate in shape. The fruit is round-oblate in shape and the calyx not prominent and appressed to the apex of the fruit. ‘Robeson’ is readily asexually propagated by either softwood or hardwood stem cuttings. It has not had any problems to date with stem blight, and is it resistant to phytophthora root rot.

With regard to consistent differences between ‘Robeson’ and ‘Premier’, internode length of ‘Roberson’ is shorter and summer stem color green verses yellow-green. The fruit also has only about ⅓ the number of viable seeds as ‘Premier’.

### BRIEF DESCRIPTION OF THE DRAWINGS

The photographs in the drawings were made using digital photography techniques, and illustrates the colors as true as reasonably possible when using these techniques. Colors in the photographs may differ slightly from the color values cited in the detailed botanical description, which accurately describe the colors of the new pentaploid hybrid Vaccinium variety. All photographs were taken from plants growing at the Castle Hayne Research Station, Castle Hayne, N.C.

FIG. 1 shows the typical plant habit of ‘Robeson’.

FIG. 2 shows the typical fruit of ‘Robeson’.

### DETAILED BOTANICAL DESCRIPTION OF THE VARIETY

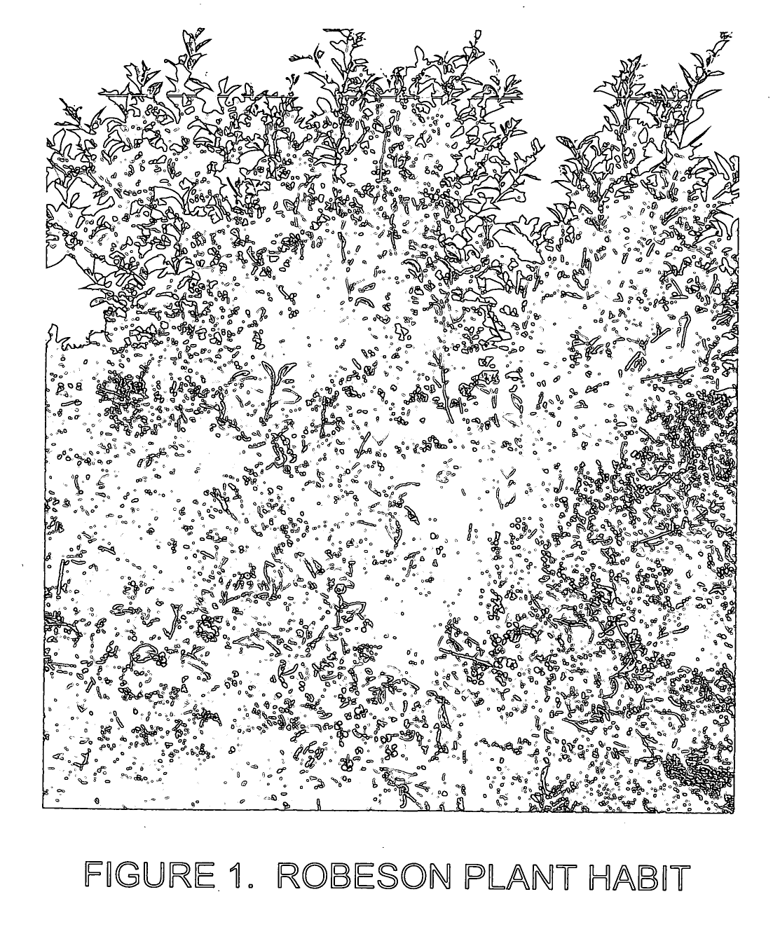

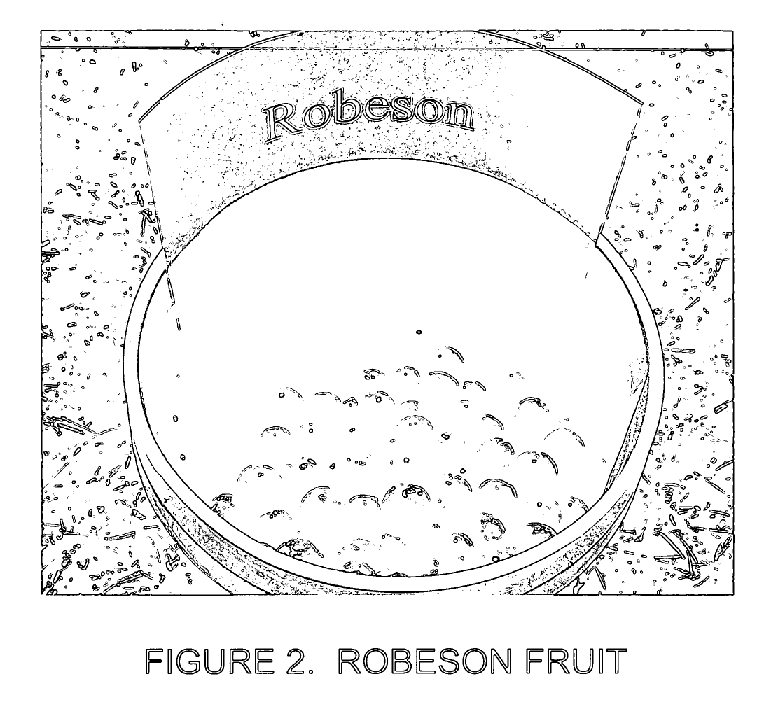

The following is a detailed botanical description of a new and distinct pentaploid hybrid variety of Vaccinium Linnaeus known as ‘Robeson’. The observations below are from mature plants grown in test plots at a standard commercial spacing of 4′ between plants in rows and 10′ between rows, at Castle Hayne, N.C. Those skilled in the art of cultivar description and evaluation will appreciate that certain characteristics of a variety will vary with older or, conversely, with younger plants. ‘Robeson’ has not been observed under all possible environmental conditions. Where dimensions, sizes, colors and other characteristics are given, it is to be understood that such characteristics are approximations or averages set forth as accurately as practicable. The phenotype of the variety may differ from the descriptions herein with variations in the environment such as season, temperature, light intensity, day length and cultural conditions. For botanical descriptive purposes ‘Robeson’ was compared to its ‘Premier’ parent. The botanical descriptive data presented are averages of data collected from mature nine year old plants growing in a replicated trial at the Castle Hayne Research Station, Castle Hayne, N.C., in 2005. The exception to using average values was with seed numbers where these were determined from a representative fruit of each cultivar.
Plant:
   Dimensions.—Robeson — 1.6 m height, 1.4 m diameter, H/D ratio 1.14. Premier — 1.9 m height, 1.5 m diameter, H/D ratio 1.27.
   Growth habit.—Upright for Robeson (FIG. 1) and Premier.
   Vigor.—Very good for Robeson and Premier.
   Plant adaptation.—Robeson and Premier are both very broadly adapted to soils including higher pH soils.
   Mature cane diameter.—4.4 cm for Robeson and Premier.
   Mature cane length.—Robeson — 1.1 m Premier — 1.5 m.
   Internode length on first flush growth.—Robeson — 11.0 cm Premier — 18.0 cm.
   Number of renewal stems.—Robeson — 1.0 Premier — 3.0.
   Dormant mature stem color.—Grayed-green (RHS 198D) for Robeson and Premier.
   Dormant one year stem color.—Robeson — red (RHS 53B) on the exposed surface, yellow-green (RHS 148B) on the unexposed surface. Premier — red (RHS 53B) on the exposed surface, green (RHS 137D) on the unexposed surface.
   First flush growth stem color in summer.—Robeson — green (RHS 138C) Premier — yellow-green (RHS 147C).
   Pubescence on summer and one year dormant stems.—No stem pubescence on Robeson or Premier.
Leaves:
   Leaf blade dimensions.—Robeson — length 81 mm, width 41 mm, L/W ratio 1.98. Premier — length 81 mm, width 34 mm, L/W ratio 2.38.
   Leaf petiole length.—Robeson — 4 mm Premier — 3 mm.
   Leaf shape.—Robeson — narrowly elliptic-obovate Premier — narrowly elliptic-obovate to oblanceolate.
   Leaf apex angle.—Robeson — acuminate to acute Premier — acuminate.
   Leaf base angle.—Acute for Robeson and Premier.
   Leaf margin.—Robeson — apical ⅔ serrulate Premier — serrulate.
   Leaf pubescence.—None for Robeson and Premier.
   Leaf glands.—Robeson — none. Premier — sparse stalked glands along lateral veins on the abaxial surface.
   Leaf color.—The adaxial surface color is green (RHS 137A) and the abaxial color green (RHS 138B) for Robeson and Premier.
Flowers:
   Number of petals.—Five, fused into a corolla tube for Robeson and Premier.
   Number of flowers per inflorescence.—Robeson — 4.0 Premier — 4.0.
   Flower dimensions.—Robeson — length 9.0 mm, diameter 5.0 mm, L/D ratio 1.8. Premier — length 9.0 mm, diameter 4.0 mm, L/D ratio 2.2.
   Length of the single style.—Robeson and Premier — 8.0 mm.
   Flower shape.—Robeson — cylindro-urceolate Premier — cylindraceous to cylindro-urceolate.
   Flower color.—Robeson — red-purple (RHS 63B) on the basal half and mainly on the petal lobes just prior to opening, fading to white (RHS 155C) when fully open. Premier — red-purple (RHS 63D) just prior to opening, fading to white (RHS 155C) on fully open flowers.
Fruit:
   Fruit dimensions.—Robeson — length 15 mm, diameter 17 mm, L/D ratio 0.88. Premier — length 15 mm, diameter 18 mm, L/D ratio 0.83.
   Fruit shape.—Robeson — round-oblate Premier — oblate.
   Fruit pedicel length.—Robeson and Premier — 8 mm.
   Fruit picking scar.—Robeson — 2.0 mm diameter, dry Premier — 1.0 mm diameter, dry.
   Fruit calyx orientation and prominence.—Appressed against the apical end of the fruit and not prominent for Robeson And Premier.
   Fruit color with bloom (epicuticular wax).—Robeson (FIG. 2) and Premier — violet-blue (RHS 97B).
   Fruit color without bloom.—Black (RHS 202A) for Robeson and Premier.
Seeds:
   Number of fully developed seeds per berry.—Robeson — 14 Premier — 46.
   Seed dimensions.—Robeson — length 1.50 mm, width 1.00 mm, L/W ratio 1.5. Premier — length 2.00 mm, width 1.00 mm, L/W ratio 2.0.
   Seed shape.—Basically depressed-ovate for Robeson and Premier. For technical (pomological) descriptive data purposes ‘Robeson’ was compared to ‘Premier’, ‘Columbus’ (unpatented) and ‘Tifblue’ (unpatented) at Castle Hayne, N.C., in Tables 2-7. The exception was for time of flowering, where the data was more representative from Jackson Springs, N.C., in 1992, and compared ‘Robeson’ with ‘Premier’ and ‘Tifblue’ (Table 1).

Time of flowering: Robeson was a few days later than Premier for date of first bloom and a few days later for date of 50% bloom (Table 1). It was just over a week earlier than Tifblue for both first bloom and 50% bloom.

TABLE 1
Time of flowering of blueberry cultivars at Jackson Springs, NC, 1992.

| Cultivar | Date of first bloom | Date of 50% bloom |
|---|---|---|
| Robeson | 3/23 | 4/11 |
| Premier | 3/21 | 4/14 |
| Tifblue | 4/1 | 4/20 |

Pollination requirements: The flowers of Robeson are not self-fertile and require cross-pollination to set commercial crops of fruit. Premier has a bloom pattern similar ro Robeson and has proved to be an excellent pollinator for this variety.
pollen production: Robeson flowers produce very little viable pollen since the variety is pentaploid.

Season of ripening: Robeson was somewhat earlier ripening than Premier and much earlier ripening than either Columbus and Tifblue (Table 2).

TABLE 2
Season of ripening for blueberry cultivars at Castle Hayne, NC.

Cumulative percent
ripe by June 29th¹

| Cultivar | 1999 | 2000 | 2001 | Average |
|---|---|---|---|---|
| Robeson | 100 | 100 | 100 | 100 |
| Premier | 86 | 100 | 93 | 93 |
| Columbus | 40 | 57 | 33 | 43 |
| Tifblue | 42 | 0 | 0 | 14 |

¹Percent ripe after the first two weeks of the season.

Yield per plant:

In 1999 and 2001 there were no significant differences between Robeson and the other three varieties for yield, and yield was quite good for all four in 2001 (Table 3). Yield in 2000 was adversely affected by the harvest method (rubber hoses) due to excessive fruit falling to the ground, so the significant differences indicated were not meaningful.

TABLE 3
Yield of blueberry cultivars at Castle Hayne, NC.

| | Yield (lbs./plant)¹ | | |
|---|---|---|---|
| Cultivar | 1999 | 2000² | 2001 |
| Robeson | 4.6 | 3.1bc | 15.2 |
| Premier | 6.6 | 4.8b | 15.8 |
| Columbus | 5.5 | 11.2a | 19.8 |
| Tifblue | 6.8 | 9.0a | 18.3 |

¹Values not followed by the same letter(s) are significantly different at the 0.05 level (Duncan's Multiple Range Test).
²Yield adversely affected by harvest method (catch frames and rubber hoses).

Fruit size (weight per berry): Fruit size of Robeson was equal to Tifblue two years out of three, and Premier one year out of three (Table 4). It had the smallest average size of the four varieties. Robeson therefore has medium size fruit in the size range generally considered suitable for mechanical harvesting.

TABLE 4
Fruit size of blueberry cultivars at Castle Hayne, NC.

Fruit size
(weight per berry in grams)¹

| Cultivar | 1999 | 2000 | 2001 | Average |
|---|---|---|---|---|
| Robeson | 1.13c | 1.46bc | 1.35c | 1.31 |
| Premier | 1.72b | 1.56b | 1.78b | 1.69 |
| Columbus | 2.07a | 2.08a | 2.12a | 2.09 |
| Tifblue | 1.64b | 1.41c | 1.36c | 1.47 |

¹Values not followed by the same letter(s) are significantly different at the 0.05 level (Duncan's Multiple Range Test).

Fruit color: In addition to The Royal Horticultural Society Colour Chart, fruit color was also determined objectively with a Minolta Color Meter (Table 5), and these data indicated in Robeson was equal to Tifblue for color, but not equal to Premier or Columbus by this means of comparison.

TABLE 5
Fruit color and fruit firmness of blueberry cultivars at Castle Hayne, NC.

| | |
|---|---|
| Color^{1,2} | Firmness^{1,3} |

| Cultivar | 2001 | 1999 | 2001 |
|---|---|---|---|
| Robeson | 17.8c | 98b | 115c |
| Premier | 20.7b | 104ab | 133b |
| Columbus | 23.4a | 102ab | 137b |
| Tifblue | 19.9bc | 115a | 175a |

¹Values not followed by the same letter(s) are significantly different at the 0.05 level (Duncan's Multiple Range Test).
²Color (lightness or “L” values) determined objectively by a Minolta Color Meter. Higher values indicate lighter blue color.
³Fruit firmness determined objectively using a Firm-tech Firmness Tester.

Fruit firmness: Fruit firmness determined by a Firm-tech Firmness Tester demonstrated that Robeson was equal to Premier and Columbus in 1999 (Table 5), but it was significantly less firm than the other three varieties in 2001. Therefore, even though fruit size of Robeson is in the appropriate range for mechanical harvest, the fruit is not sufficiently firm to mechanically harvest for the fresh market, and would only be appropriate for processing markets following mechanical harvesting, which is not high priority in North Carolina.

Fruit flavor: Subjective ratings to flavor indicated that Robeson consistently scored in the very good range (table 6), and fruit quality is definitely one of this variety's strong points. It was significantly better than Premier in all three years, and equal to or better than Tifblue all three years. It was only equal to Columbus one year out of three.

TABLE 6
Fruit flavor of blueberry cultivars at Castle Hayne, NC.

| | Flavor^{1,2} | | |
|---|---|---|---|
| Cultivar | 1999 | 2000 | 2001 |
| Robeson | 79a | 77bc | 78b |
| Premier | 75c | 75d | 77c |
| Columbus | 79a | 79a | 80a |
| Tifblue | 77b | 77bc | 78b |

¹Values not followed by the same letter(s) are significantly different at the 0.05 level (Duncan's Multiple Range Test).
²Subjective ratings based on a 0-90 scale, where less than 60 is unsatisfactory, 60-69 is satisfactory, 70-79 is average to good, and 80 and above superior.

Post harvest shelf-life: Post harvest shelf-life of fruit is not one of the strongest characteristics of Robeson (Table 7). It was significantly poorer than the other three varieties following seven days storage at both temperatures. Therefore it is most strongly recommended for production for local direct markets

TABLE 7
Post harvest shelf-life of the fruit of blueberry cultivars at Castle Hayne,
NC, in 2001.

Percent marketable fruit after seven days¹

| Cultivar | 50° F. | 70° F. |
|---|---|---|
| Robeson | 46c | 10c |
| Premier | 70b | 25b |
| Columbus | 84a | 69a |
| Tifblue | 81a | 77a |

¹Values not followed by the same letter(s) are significantly different at the 0.05 level (Duncan's Multiple Range Test).

Propagation: Robeson is easily propagated asexually by both hardwood and softwood stem cuttings.
Disease reaction: Robeson has not had any problems with stem blight (Botryospaeria dothidea) one of the two major diseases affecting blueberries in North Carolina. It is susceptible to the other major disease, steam canker (Botryosphaeria corticis). It is also resistant to phytophthora root rot (Phytophthora cinnamoni).
Herbarium voucher: A voucher of Robeson will be deposited in the Herbarium of North Carolina State University (NCSU) in Raleigh, N.C., USA upon patenting.

That which claimed is:

1. A new and distinct variety of commercial blueberry (Vaccinium×‘Robeson’) substantially as illustrated and described, characterized by its broad soil adaptation, early ripening, high yields, very good fruit quality, medium fruit size, limited numbers of seeds per fruit, and adaptation to direct market outlets.
